(19) United States
(12) Patent Application Publication
(10) Pub. No.: US 2005/0087196 A1
(43) Pub. Date: Apr. 28, 2005

(54) Patient restraint strap

(76) Inventor: Lorie Lynn Deem-Rockstroh, Canton, OH (US)

Correspondence Address: Zollinger & Burleson, Ltd., P.O. Box 2368, North Canton, OH, 44720, US

(21) Appl. No.: 10/966,336

(22) Filed: Oct. 16, 2004

Related U.S. Application Data
(60) Provisional application No. 60/514,103, filed on Oct. 27, 2003.

(51) Int. Cl. A61B 19/00

(52) U.S. Cl. 128/869

(57) ABSTRACT

A patient restraint strap for securing a patient to a backboard generally includes a first strap and a second strap that are connected to opposite sides of the backboard. Both straps includes first and second strips that are securely connected together to form an integral strap. The first strip is used to connect the strap to the backboard. The second strips are at least 4 inches wide so that the load of the strap is spread over a large area of the patient. The second strips also have the connectors used to secure the straps together. The connectors and method of using the strap eliminates undesirable cinching and allows the strap to be readily adjusted for a wide variety of patient sizes. The strap is also configured to be comfortable for the patient.

### CROSS REFERENCE TO RELATED APPLICATIONS

This application claims priority from U.S. Provisional Patent Application Ser. No. 60/514,103 filed Oct. 27, 2003; the disclosure of which is incorporated herein by reference.

### BACKGROUND OF THE INVENTION

1. Technical Field

The present invention generally relates to patient restraint straps and, more particular, to patient restraint straps used to secure patients to backboards. The invention also relates to a method of securing a patient to a backboard with a restraint strap.

2. Background Information

A person with a head, neck or back injury generally requires some degree of immobilization until the person is delivered to an emergency facility for higher medical attention. In order to immobilize a patient, the patient is placed supine on a flat-rigid board running the entire length of his body. The patient's head is secured to the board with a head immobilizer. The patient's body is secured to the board by placing straps across his chest, hip, and lower extremities. The board used to achieve spinal immobilization is known as a backboard. The backboard includes several opposed openings on both long sides of the board to accommodate the straps that secure the patient to the board. These openings are provided in two different styles. The first type of opening has a pin through the middle which allows a strap to be connected with a first style of quick-connect fastener. The second type of opening has no pin and allows for a strap to be connected with a second style of quick-connect fastener. The straps used to secure the patient to the backboard are two-piece straps that connect together with a snap buckle such as the buckles used with most seat belts in automobiles. The straps themselves are fabricated from a tough woven nylon similar to that used with automobile seatbelts. These straps are 2 inches wide. EMS workers have problems adequately immobilizing patients and certain injuries with these narrow straps. The straps have been found to create undesirable pressure points and pinch the patients.

Typical two-piece straps are connected with plastic or metal buckles. One problem with buckles is that the buckles create a bulge at the location of the buckle. The bulge undesirable presses against the patient. When the buckles are fabricated from plastic, the plastic eventually becomes brittle and can fail. When the buckles are metal, they must be disconnected and moved out of the way before X-rays are taken. Buckles are also difficult to clean when they become saturated with bodily fluids. Most prior art buckles work by allowing a portion of the strap to slide through the buckle to adjust the belt. This configuration forces the belt to be cinched against the patient for the strap to be tightened. The cinching action is uncomfortable for the patient. The cinching action is also difficult for the EMS workers. In order to tighten a strap with a sliding buckle, the EMS worker must pull the loose end of the strap substantially parallel to the portion of the strap being tightened. Pulling in this direction can be awkward. Strap cinching also can cause a worker to over-tighten a strap.

### BRIEF SUMMARY OF THE INVENTION

In one embodiment, the invention provides a strap for restraining a patient on a backboard. The strap includes a first strap having inner and outer strips with each of the inner and outer strips having a first end and a second end. The inner and outer strips of the first strap are securely fastened together. The inner strip of the first strap has an inner surface and an outer surface. The outer strip of the first strap is connected to the outer surface of the inner strip of the first strap. The strap also has a second strap having inner and outer strips with each of the inner and outer strips having a first end and a second end. The inner and outer strips of the second strap are securely fastened together. The inner strip of the second strap has an inner surface and an outer surface with the outer strip of the second strap being connected to the outer surface of the inner strip of the second strap. Each of the inner strips is at least 4 inches wide. A section of hooks of a hook and loop fastener is connected to the inner surface of the inner strip of the first strap and a section of loops of a hook and loop fastener is connected to the inner surface of the inner strip of the second strap.

The invention also provides a method for installing the restraint strap wherein the second strap is doubled over itself with a portion of the loops facing the patient and a portion of the loops facing outwardly from the patient. The first strap is disposed over top of the portion of the second strap having the loops facing outwardly from the patient with the hooks of the first strap engaging the loops of the second strap.

In another embodiment, the invention provides a method for installing the restraint strap wherein the first strap is disposed over the patient with the section of hooks facing outwardly away from the patient. The second strap is disposed over the top of the first strap with the loops facing the patient and a portion of the loops engaging the hooks of the first strap.

### BRIEF DESCRIPTION OF THE SEVERAL VIEWS OF THE DRAWINGS

FIG. 1 is a perspective view of a patient secured to a backboard with three of the restraint straps of the invention.

FIG. 2 is a top plan view of a typical backboard.

FIG. 3 is a side view of a first embodiment of a quick-connect fastener used to secure one end of the restraint strap to the backboard.

FIG. 4 is a top plan view of the quick-connect fastener of FIG. 3 connected to the backboard.

FIG. 5 is a top plan view of a second embodiment of a quick-connect fastener shown being used to secure one end of the restraint strap to the backboard.

FIG. 6 is a top plan view of the first strap of the restraint strap of the invention used with the second embodiment of the quick-connect fastener.

FIG. 7 is a side view of the first strap of the restraint strap shown in FIG. 6.

FIG. 8 is a bottom plan view of the first strap of the restraint strap shown in FIG. 6.

FIG. 9 is a top plan view of the first strap of the restraint strap of the invention used with the first embodiment of the quick-connect fastener.

FIG. 10 is a side view of the first strap of the restraint strap shown in FIG. 9.

FIG. 11 is a bottom plan view of the first strap of the restraint strap shown in FIG. 9.

FIG. 12 is a top plan view of the second strap of the restraint strap of the invention used with the second embodiment of the quick-connect fastener.

FIG. 13 is a side view of the second strap of the restraint strap shown in FIG. 12.

FIG. 14 is a bottom plan view of the second strap of the restraint strap shown in FIG. 12.

FIG. 15 is a top plan view of the second strap of the restraint strap of the invention used with the first embodiment of the quick-connect fastener.

FIG. 16 is a side view of the second strap of the restraint strap shown in FIG. 15.

FIG. 17 is a bottom plan view of the second strap of the restraint strap shown in FIG. 15.

FIG. 18 is a schematic end view showing one configuration for the use of the restraint strap.

FIG. 19 is a schematic end view showing a second configuration for the use of the restraint strap.

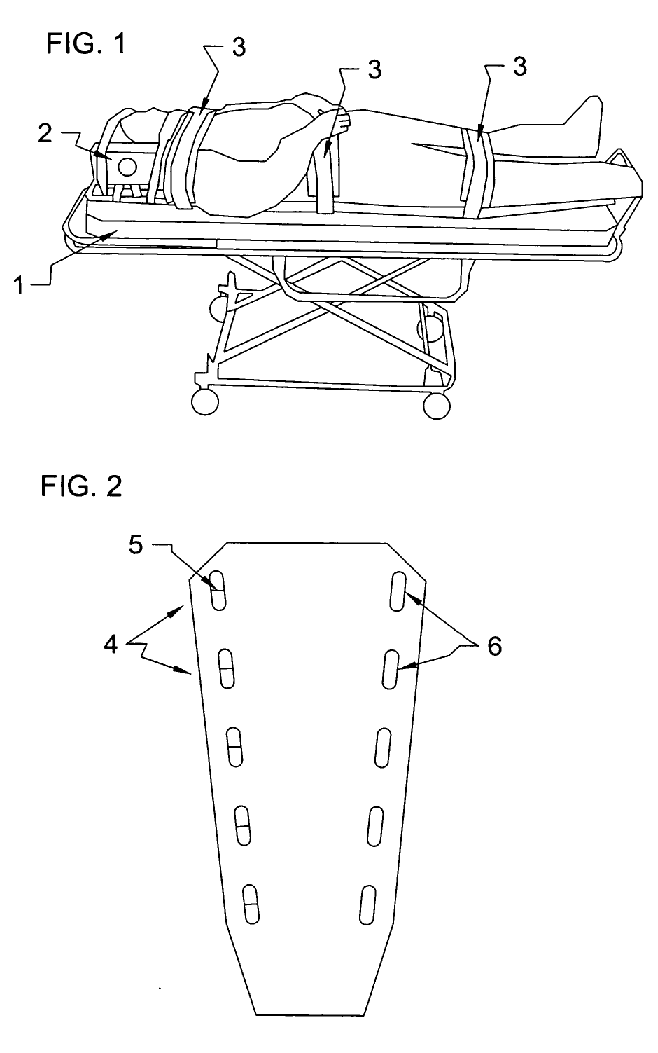

Similar numbers refer to similar parts throughout the specification.

### DETAILED DESCRIPTION OF THE INVENTION

An exemplary embodiment of the patient restraint strap of the invention is indicated generally by the numeral 3 in the accompanying drawings. Strap 3 generally includes a first strap 50 and a second strap 52 that cooperate with each other to secure a patient to a backboard 1 with or without head restraint 2. Strap 3 is designed to secure a patient on backboard 1 without undesirable cinching and over-tightening. Strap 3 is also designed to be comfortable for the patient while being fully washable and invisible to X-rays. Further, strap 3 is designed to be comfortable for the patient.

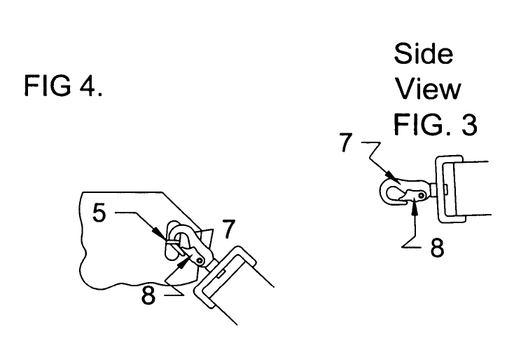

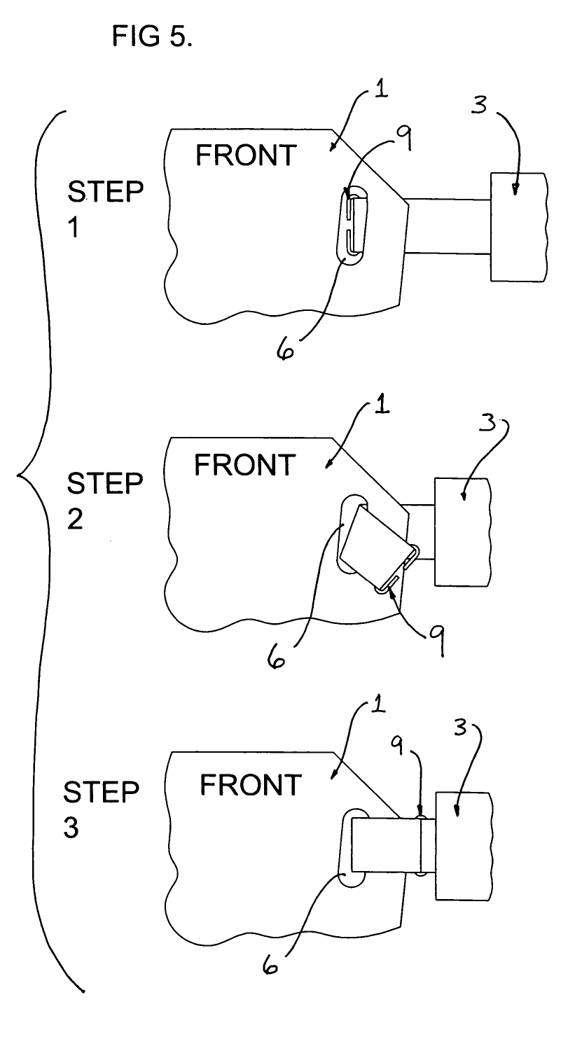

FIG. 2 shows a schematic view of a typically backboard 1 except that different openings 4 and 6 are shown disposed on opposite side of backboard 1 (the openings would usually be the same on the opposite sides of backboard 1). Opening 4 is the type having a pin 5 disposed across opening 4 while opening 6 is an unobstructed opening. Strap 3 is shown in this application used with fasteners 7 and 9 adapted to work with both types of openings 4 and 6. FIGS. 3 and 4 show how fastener 7 allows strap 3 to be connected directly to pin 5 and locked in place with a latch 8. FIG. 5 shows how strap 5 is looped through opening 6 and clipped to itself with fastener 9. Strap 3 is looped by passing its leading end under the edge of backboard 1 so that the body of strap 3 is disposed outwardly of backboard 1. The user then fits a portion of strap 3 through the slit opening of fastener 9 to lock strap 3 to backboard 1 as shown in Step 3 of FIG. 5.

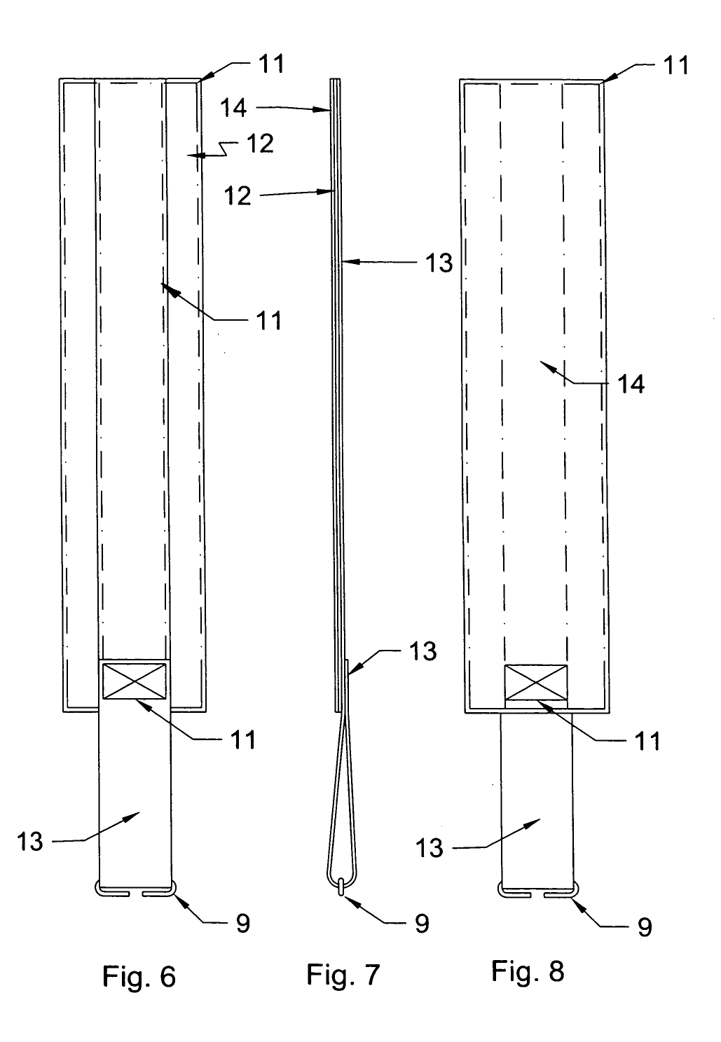

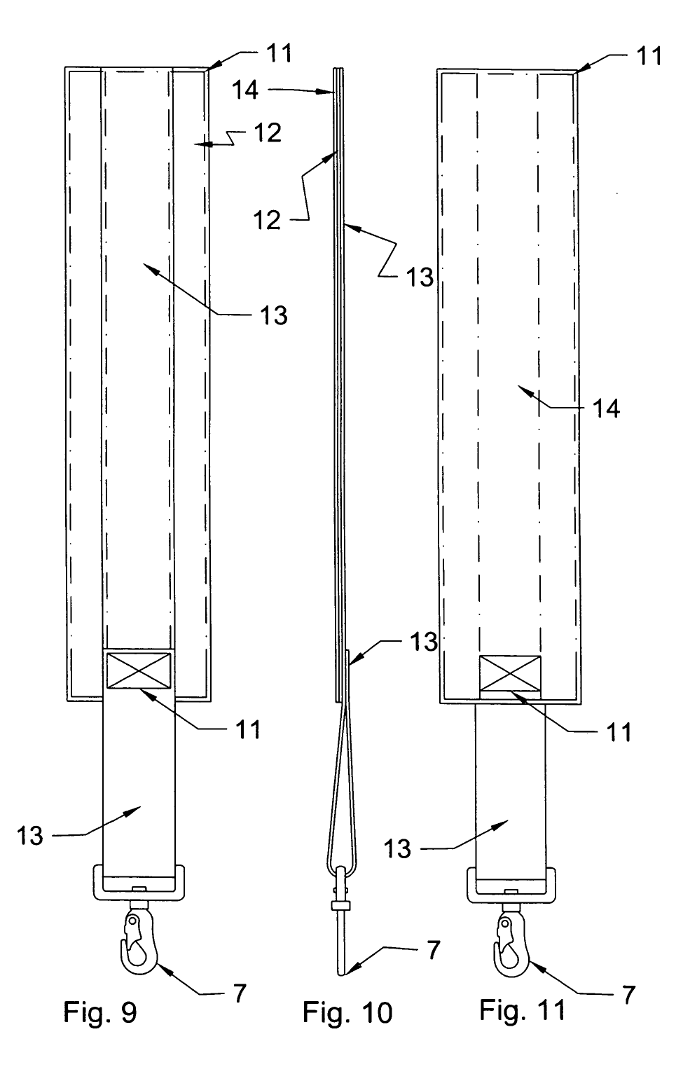

FIGS. 6 through 11 illustrate exemplary embodiments of first strap 50. First strap 50 includes a wide inner strip 12 having an inner end 54 and an outer end 56. First strap 50 also includes a thin outer strip 13 having an inner end 58 and an outer end 60. Strips 12 and 13 are longitudinally centered (as shown in FIGS. 6 and 9) and securely fastened together with inner ends 54 and 58 aligned. Any of a variety of known fasteners may be used to fasten strips 12 and 13 together with stitching 11 being shown in the exemplary drawings. Outer end 60 of outer strip 13 extends past outer end 56 of inner strip 12. Outer end 60 carries fastener 7 or 9. Outer end 60 is the outermost portion of a loop formed by doubling outer strip 13 back on itself. In the exemplary embodiment, stitches 11 are used to form the loop with stitches 11 extending entirely through outer strip 13 and inner strip 11 as shown in FIGS. 6, 8, 9, and 11.

Outer strip 13 may be fabricated from a tough, washable Nylon webbing such as that typically used in automotive seat belts. Inner strip 12 may be fabricated from Vinyl. A section of hooks 14 of hook and loop mechanical fasteners is disposed on the inwardly facing surface of inner strip 12 and may cover the entire inwardly facing surface as shown in FIGS. 8 and 11. The outer surface of inner strip 12 may be provided with a bright or reflective material so that straps 3 may be readily seen in low light situations and so that a patient strapped to a backboard may be readily seen in low-light situations. All of these materials are washable and do not interfere with X-rays.

Inner strip 12 of first strap 50 is at least 4 inches wide and 18 inches long. Outer strip 13 of first strap 50 is at least 2 inches and is long enough to have outer end 60 extend at least ¾ inch past outer end 56.

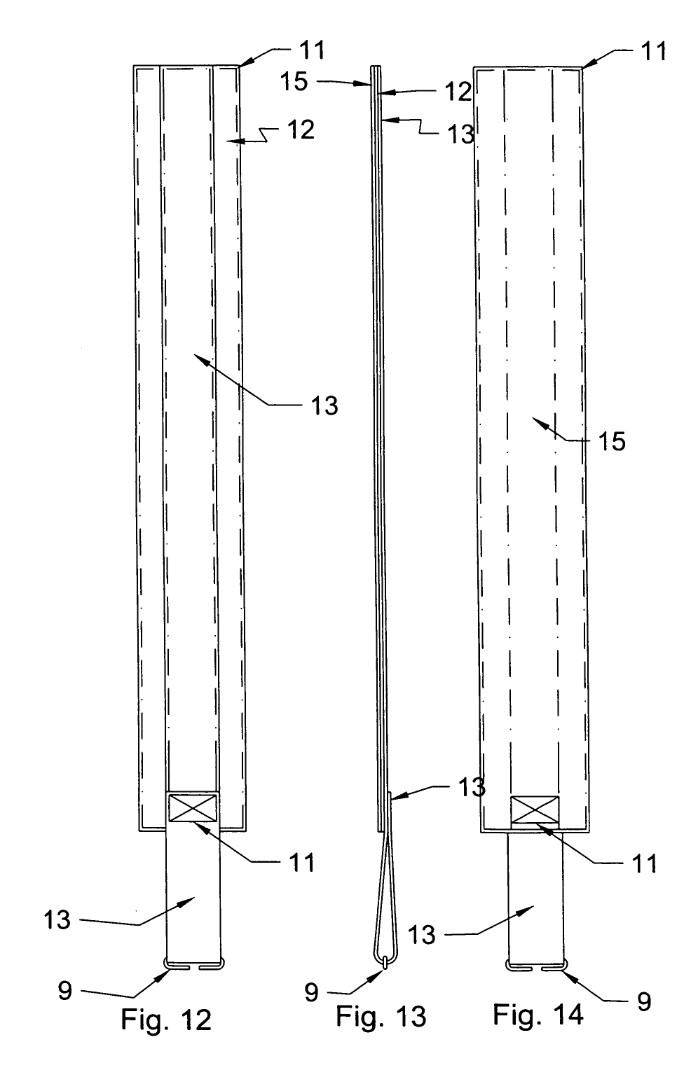

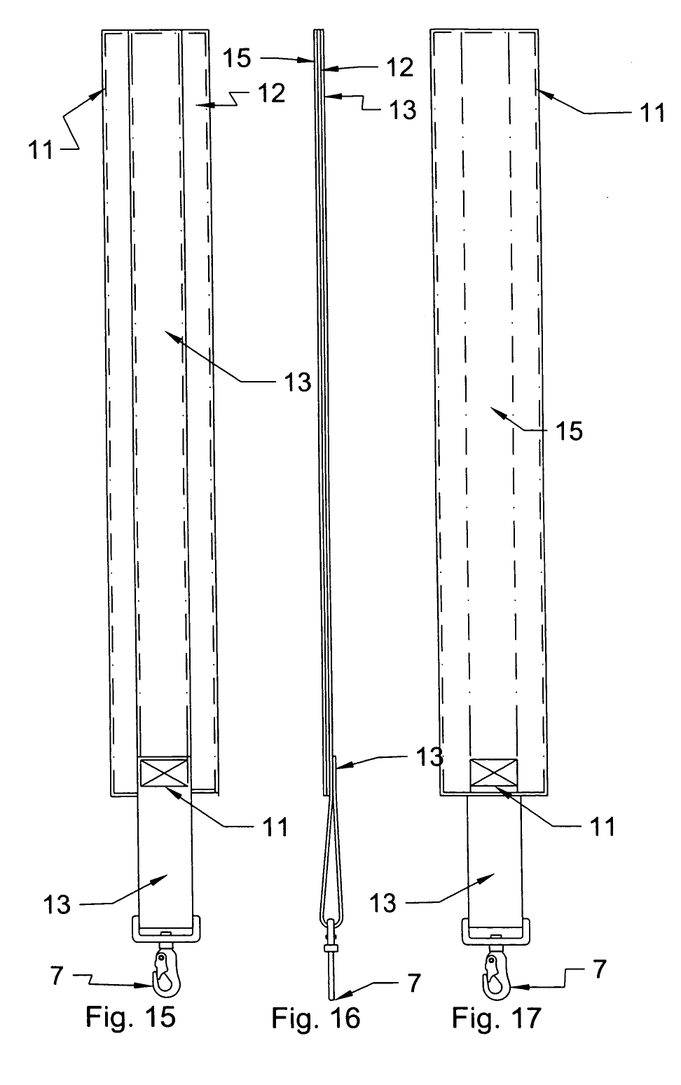

FIGS. 12 through 17 illustrate exemplary embodiments of second strap 52. Second strap 52 includes a wide inner strip 12 having an inner end 74 and an outer end 76. Second strap 52 also includes a thin outer strip 13 having an inner end 78 and an outer end 80. Strips 12 and 13 are longitudinally centered (as shown in FIGS. 12 and 15) and securely fastened together with inner ends 74 and 78 aligned. Any of a variety of known fasteners may be used to fasten strips 12 and 13 together with stitching 11 being shown in the exemplary drawings. Outer end 80 of outer strip 13 extends past outer end 76 of inner strip 12. Outer end 80 carries fastener 7 or 9. Outer end 80 is the outermost portion of a loop formed by doubling outer strip 13 back on itself. In the exemplary embodiment, stitches 11 are used to form the loop with stitches 11 extending entirely through outer strip 13 and inner strip 11 as shown in FIGS. 12, 14, 15, and 17.

Outer strip 13 may be fabricated from a tough, washable Nylon webbing such as that typically used in automotive seat belts. Inner strip 12 may be fabricated from Vinyl. A section of loops 15 of hook and loop mechanical fasteners is disposed on the inner surface of inner strip 12 and may cover the entire inwardly facing surface as shown in FIGS. 14 and 17. The outer surface of inner strip 12 may be provided with a bright or reflective material so that straps 3 may be readily seen in low light situations and so that a patient strapped to a backboard may be readily seen in low-light situations. All of these materials are washable and do not interfere with X-rays.

Inner strip 12 of second strap 52 is at least 4 inches wide and 29 inches long. Outer strip 13 of second strap 52 is at least 2 inches and is long enough to have outer end 60 extend at least ¾ inch past outer end 56.

The use of the 4 inch inner strips 12 has been found to be useful for immobilizing a patient while also being useful for stabilizing an injury such as a bone fracture. The width allows the EMS workers to immobilize large bone fractures with strap 3 instead of being forced to use a thin strap in combination with a splinting device. Strap 3 thus saves time in stabilizing the patient and delivering the patient to the medical facility. The 4 inch wide inner strips 12 have also been found to be desirable because they spread the load of strap 3 over a wider area for the comfort of the patient.

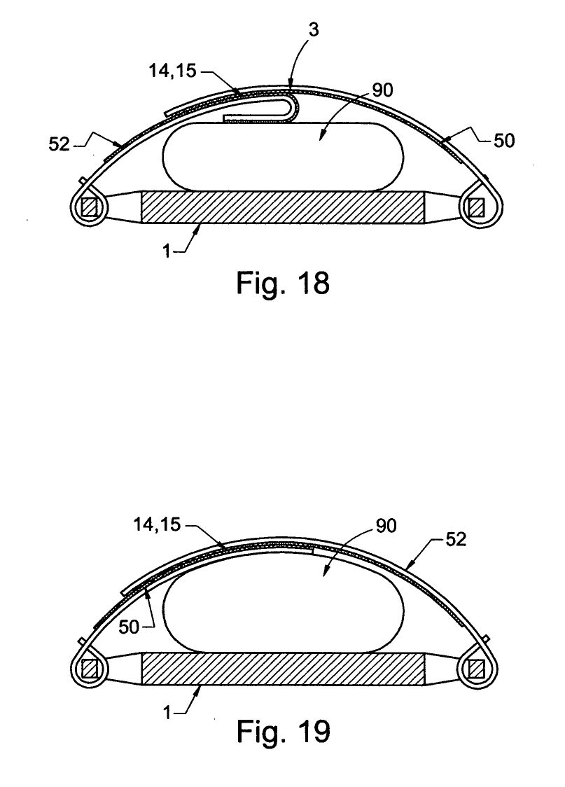

In one embodiment of the invention, strap 3 is installed as shown in FIG. 18 with second strap 52 being doubled over itself with a portion of the loops 15 facing the patient 90 and a portion of loops 15 facing outwardly from patient 90. First strap 50 is then disposed over top of the portion of the second strap 52 having the loops facing outwardly from the patient with the hooks of the first strap engaging the loops of the second strap. This configuration provides a comfort to the patient because the loops of the hook and loop connector engage the patient instead of the hooks which can irritate the skin. This configuration is also desirable because there are no loose ends.

In another embodiment of the invention, strap 3 is installed as shown in FIG. 19 with first strap 50 disposed over patient 90 with section of hooks 14 facing outwardly away from patient 90. Second strap 52 is disposed over the top of first strap 50 with loops 15 facing patient 90 and a portion of loops 15 engaging hooks 14 of first strap 50. In this configuration, no portion of hooks 14 are exposed to patient 90 thus eliminating and discomfort caused by hooks 14.

Both of these strapping methods do not require the undesirable cinching of prior art straps. Both of these configurations also engage the hook side of the hook and loop fastener to the patient to create friction between strap 3 and patient 90 to help stabilize patient 90 with respect to strap 3 and backboard 1. Both strap configurations may be used without the need for positioning portions of strap 3 under the patient. Strap 3 may this be used after a patient is positioned on board 1 without the need to move the patient.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A strap for restraining a patient on a backboard; the strap comprising:
a first strap having inner and outer strips; each of the inner and outer strips having an inner end and an outer end; the inner and outer strips of the first strap being securely fastened together;
the inner strip of the first strap having an inner surface and an outer surface; the outer strip of the first strap being connected to the outer surface of the inner strip of the first strap;
a second strap having inner and outer strips; each of the inner and outer strips having an inner end and an outer end; the inner and outer strips of the second strap being securely fastened together;
the inner strip of the second strap having an inner surface and an outer surface; the outer strip of the second strap being connected to the outer surface of the inner strip of the second strap;
each of the inner strips being at least 4 inches wide;
a section of hooks of a hook and loop fastener being connected to the inner surface of the inner strip of the first strap; and
a section of loops of a hook and loop fastener being connected to the inner surface of the inner strip of the second strap.

2. The strap of claim 1, wherein each of the outer strips is less than 4 inches wide.

3. The strap of claim 2, wherein each of the outer strips is 2 inches wide.

4. The strap of claim 1, wherein the inner and outer strips are stitched together.

5. The strap of claim 1, wherein each of the outer strips is fabricated from Nylon.

6. The strap of claim 5, wherein each of the inner strips is fabricated from Vinyl.

7. The strap of claim 1, wherein the section of hooks covers the entire inner surface of the inner strip of the first strap and the section of hooks covers the entire inner surface of the inner strip of the second strap.

8. The strap of claim 1, further comprising a fastener connected to each of the outer strips.

9. The strap of claim 8, wherein each of the outer strips is looped back on itself to form a loop; the fastener being connected to the loop.

10. The strap of claim 1, wherein the inner ends of the first and second strips are aligned.

11. A strap for restraining a patient on a backboard; the strap comprising:
a first strap having inner and outer strips; each of the inner and outer strips having an inner end and an outer end; the inner and outer strips of the first strap being securely fastened together;
the outer end of the first strap being adapted to be connected to the backboard;
the inner strip of the first strap having an inner surface and an outer surface; the outer strip of the first strap being connected to the outer surface of the inner strip of the first strap;
a second strap having inner and outer strips; each of the inner and outer strips having an inner end and an outer end; the inner and outer strips of the second strap being securely fastened together;
the outer end of the second strap being adapted to be connected to the backboard;
the inner strip of the second strap having an inner surface and an outer surface; the outer strip of the second strap being connected to the outer surface of the inner strip of the second strap;
each of the inner strips being at least 4 inches wide;
a section of hooks of a hook and loop fastener being connected to the inner surface of the inner strip of the first strap;
a section of loops of a hook and loop fastener being connected to the inner surface of the inner strip of the second strap;
the second strap being doubled over itself with a portion of the loops facing the patient and a portion of the loops facing outwardly from the patient; and
the first strap being disposed over top of the portion of the second strap having the loops facing outwardly from the patient with the hooks of the first strap engaging the loops of the second strap.

12. The strap of claim 11, wherein each of the outer strips is less than 4 inches wide.

13. The strap of claim 12, wherein each of the outer strips is 2 inches wide.

14. The strap of claim 11, wherein the section of hooks covers the entire inner surface of the inner strip of the first strap and the section of hooks covers the entire inner surface of the inner strip of the second strap.

15. The strap of claim 11, further comprising a fastener connected to each of the outer strips.

16. The strap of claim 15, wherein each of the outer strips is looped back on itself to form a loop; the fastener being connected to the loop.

17. A strap for restraining a patient on a backboard; the strap comprising:
a first strap having inner and outer strips; each of the inner and outer strips having an inner end and an outer end; the inner and outer strips of the first strap being securely fastened together;
the outer end of the first strap being adapted to be connected to the backboard;
the inner strip of the first strap having an inner surface and an outer surface; the outer strip of the first strap being connected to the outer surface of the inner strip of the first strap;
a second strap having inner and outer strips; each of the inner and outer strips having an inner end and an outer end; the inner and outer strips of the second strap being securely fastened together;
the outer end of the second strap being adapted to be connected to the backboard;
the inner strip of the second strap having an inner surface and an outer surface; the outer strip of the second strap being connected to the outer surface of the inner strip of the second strap;
each of the inner strips being at least 4 inches wide;
a section of hooks of a hook and loop fastener being connected to the inner surface of the inner strip of the first strap;
a section of loops of a hook and loop fastener being connected to the inner surface of the inner strip of the second strap;
the first strap being disposed over the patient with the section of hooks facing outwardly away from the patient; and
the second strap disposed over the top of the first strap with the loops facing the patient and a portion of the loops engaging the hooks of the first strap.

18. The strap of claim 17, wherein each of the outer strips is less than 4 inches wide.

19. The strap of claim 18, wherein each of the outer strips is 2 inches wide.

20. The strap of claim 17, wherein the section of hooks covers the entire inner surface of the inner strip of the first strap and the section of hooks covers the entire inner surface of the inner strip of the second strap.

21. The strap of claim 17, further comprising a fastener connected to each of the outer strips.

22. The strap of claim 21, wherein each of the outer strips is looped back on itself to form a loop; the fastener being connected to the loop.
